(19) United States
(12) Patent Application Publication
(10) Pub. No.: US 2009/0318714 A1
(43) Pub. Date: Dec. 24, 2009

(54) ORGANIC COMPOUNDS

(76) Inventors: John Mykytiuk, Oxfordshire (GB); Ludovic Bonnet, Oxfordshire (GB); Stephen Gorsuch, Oxfordshire (GB); Osamu Ichihara, Oxfordshire (GB); Richard Mears, Oxfordshire (GB); Christine Richardson, Basel (CH)

Correspondence Address: DILWORTH & BARRESE, LLP, 1000 WOODBURY ROAD, SUITE 405, WOODBURY, NY, 11797, US

(21) Appl. No.: 12/091,574

(22) PCT Filed: Nov. 6, 2006
(86) PCT No.: PCT/EP06/10612

§ 371 Date: Sep. 17, 2008

(30) Foreign Application Priority Data
Nov. 8, 2005 (GB) 0522789.7

(51) Int. Cl. C07D 309/30; C07C 229/34

(52) U.S. Cl. 549/293; 562/442

(57) ABSTRACT

The invention related to a novel process, novel process steps and novel intermediates useful in the synthesis of pharmaceutically active compounds, especially renin inhibitors, such as Aliskiren. Inter alia, the invention relates to a process for the manufacture of a compound of the formula VI,

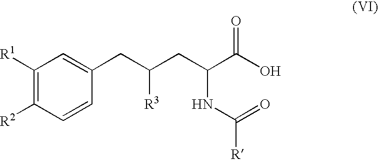

or a salt thereof, wherein R¹, R², R³ and R′ are as defined in the specification, and processes of manufacturing this compound including intermediates.

### FIELD OF THE INVENTION

The present invention relates to novel methods for preparing aryl amino acid compounds. Moreover, the present invention relates to the intermediates of the methods for preparing these compounds.

These aryl amino acid compounds are more specifically N-substituted 2-amino-4-alkyl-5-arylpentanoic acids according to formula (VI) as shown below. Such compounds are key intermediates in the preparation of renin inhibitors, in particular 2(S),4(S),5(S),7(S)-2,7-dialkyl-4-hydroxy-5-amino-8-aryl-octanoyl amide derivatives, or pharmaceutically acceptable salts thereof. Therefore, the present invention is also directed to useful intermediates in the preparation of these renin inhibitors as well as methods for preparing these intermediates.

### BACKGROUND OF THE INVENTION

Renin passes from the kidneys into the blood where it affects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume whose increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

With compounds such as (with INN name) aliskiren (2S,4S,5S,7S)-5-amino-N-(2-carbamo-yl-2-methylpropyl)-4-hydroxy-2-isopropyl-7-[4-methoxy-3-(3-methoxypropoxy)benzyl]-8-methylnonanamide), a new antihypertensive has been developed which interferes with the renin-angiotensin system at the beginning of angiotensin II biosynthesis.

As the compound comprises 4 chiral carbon atoms, the synthesis of the enantiomerically pure compound is quite demanding. Therefore, amended routes of synthesis that allow for more convenient synthesis of this sophisticated type of molecules are welcome.

Such (2S,4S,5S,7S)-2,7-dialkyl-4-hydroxy-5-amino-8-aryl-octanoyl amide derivatives are any of those having renin inhibitory activity and, therefore, pharmaceutical utility and include, e.g., those disclosed in U.S. Pat. No. 5,559,111. To date, various methods of preparing (2S,4S,5S, 7S)-2,7-dialkyl-4-hydroxy-5-amino-8-aryl-octanoyl amide derivatives are described in the literature.

In EP-A-0678 503, δ-amino-γ-hydroxy-ω-aryl-alkanecarboxamides are described, which exhibit renin-inhibiting properties and could be used as antihypertensive agents in pharmaceutical preparations.

In WO 02/02508, a multistep manufacturing process to obtain δ-amino-γ-hydroxy-ω-aryl-alkanecarboxamides is described, in which the central intermediate is a 2,7-dialkyl-8-aryl-4-octenic acid or a 2,7-dialkyl-8-aryl-4-octenic acid ester. The double bond of this intermediate is simultaneously halogenated in the 4/5 position and hydroxylated in the 4-position via (under) halo-lactonisation conditions. The halolactone is converted to a hydroxy lactone and then, the hydroxy group is converted into a leaving group, which is substituted with azide, the lactone amidated and then the azide is converted into the amine group.

Further processes for the preparation of intermediates to manufacture δ-amino-γ-hydroxy-ω-aryl-alkanecarboxamides are described in WO02/092828 (pertaining to the preparation of 2-alkyl-5-halogenpent-4-ene carboxylic esters), WO 2001/009079 (pertaining to the preparation of 2-alkyl-5-halogenpent-4-ene carboxylic acids), WO 02/08172 (pertaining to the preparation of 2,7-dialkyl-4-hydroxy-5-amino-8-aryloctanoyl amides), WO 02/02500 (pertaining to 2-alkyl-3-phenylpropionic acids), and WO02/024878 (pertaining to 2-alkyl-3-phenylpropanols).

Methods of preparing N-substituted 2-amino-4-alkyl-5-arylpentanoic acids and its derivatives are disclosed e.g. in Helv. Chim. Act., 2003, 86, 8, 2848-2870, where

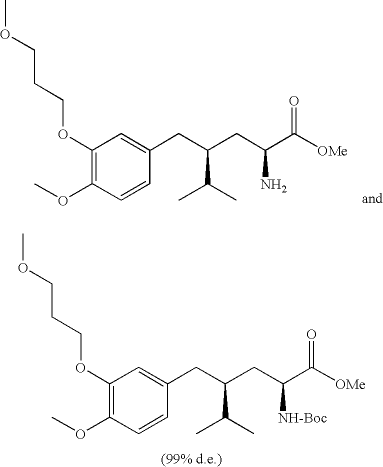

are prepared in 12 and 13 synthetic steps respectively; and in Tet. Lett., 2005, 46, 6337-6340, where

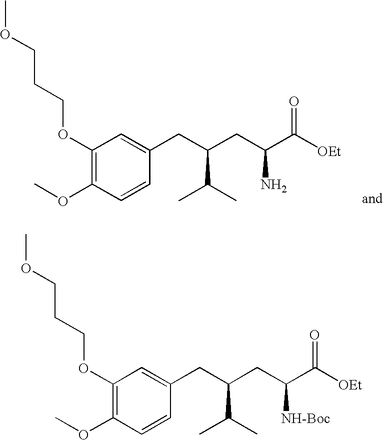

are prepared in 9 and 10 synthetic steps respectively and although

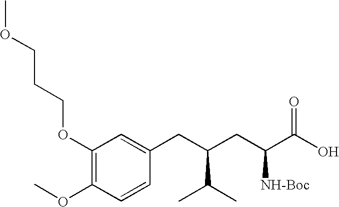

is not isolated, it is used as an intermediate and it is prepared in 11 synthetic steps.

In EP-A-1215201 an alternative route to obtain δ-amino-γ-hydroxy-ω-aryl-alkanecarboxamides is disclosed. In PCT application EP2005/009347 (WO 2006/024501) methods of preparing amino-γ-hydroxy-ω-aryl-alkanecarboxamides are described starting from L-pyro-glutamic acid and using an N-substituted 2-amino-4-alkyl-5-arylpentanoic acid as an intermediate. Although this method has certain advantages, the preparation of the N-substituted 2-amino-4-alkyl-5-arylpentanoic acid intermediate requires a number of steps and can be further improved.

Although the existing processes may lead to the desired renin inhibitors, in particular the (2S,4S,5S,7S)-2,7-dialkyl-4-hydroxy-5-amino-8-aryl-octanoyl amide derivatives, there is a need to provide an alternative synthetic route to these (2S,4S,5S,7S)-2,7-dialkyl-4-hydroxy-5-amino-8-aryl-octanoyl amide derivatives to ensure its manufacture in a simple and efficient manner.

### SUMMARY OF THE INVENTION

Surprisingly, it has now been found that renin inhibitors, in particular (2S,4S,5S,7S)-2,7-dialkyl-4-hydroxy-5-amino-8-aryl-octanoyl amide derivatives, are obtainable in high diastereomeric and enantiomeric purity and in an economic manner by using a N-substituted 2-amino-4-alkyl-5-arylpentanoic acid as an intermediate. In particular, it was found that by using a novel process and novel intermediates to prepare the N-substituted 2-amino-4-alkyl-5-arylpentanoic acid, the steps for the total synthesis of renin inhibitors, in particular (2S,4S,5S,7S)-2,7-dialkyl-4-hydroxy-5-amino-8-aryl-octanoyl amide derivatives, are considerably reduced and improved, so that the process is more economic than the prior art processes. The use of a N-substituted 2-amino-4-alkyl-5-arylpentanoic acid as an intermediate and an improved process of obtaining same, thus, simplifies the method of preparing such sophisticated types of molecules.

### DETAILED DESCRIPTION OF THE INVENTION

In a first aspect, the present invention relates to a method for the preparation of a compound of the formula (VI)

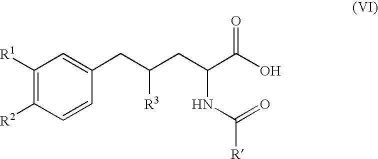

wherein
R¹ is hydrogen, halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof;
said method comprising hydrogenation of a pyrone compound of formula (V)

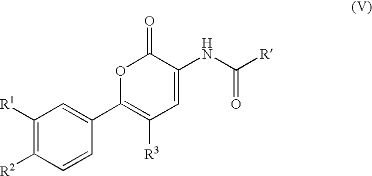

wherein R¹, R², R³ and R′ are as defined for formula (VI), or a salt thereof, to effect ring opening.

The hydrogenation preferably takes place under conditions so as to keep the other functionalities on the molecule intact by using methods well known to the person skilled in the art. Hydrogenation typically takes place in the presence of a catalyst selected from a heterogeneous catalyst or a homogeneous catalyst, such as Wilkinson's catalyst, preferably a heterogeneous catalyst. Examples of the catalyst include Raney nickel, palladium/C, Pd(OH)₂ (Perlman's catalyst), nickel boride, platinum metal or platinum metal oxide, rhodium, ruthenium and zinc oxide, more preferably palladium/C, platinum metal or platinum metal oxide, most preferably palladium/C. When palladium/C is employed, it is preferably used as a wet paste, more preferably as a 40-60% wet paste. The catalyst is preferably used in an amount of 1 to 20 mol %, more preferably 5 to 10 mol %. The reaction can be conducted at atmospheric or elevated hydrogen pressure, such as a pressure of 2-12 bar, e.g. 5-10 bar, more preferably 8 bar. It is preferred to conduct the reaction at elevated hydrogen pressure. The hydrogenation takes place preferably in an inert solvent typically employed in a hydrogenation, more preferably in an alcoholic solvent such as methanol, ethanol, n-propanol, isopropanol, n-butanol, sec-butanol and isobutanol, preferably ethanol, isopropanol, sec-butanol or n-butanol, most preferably sec-butanol, and also mixtures of these solvents with water are possible. The reaction time and the temperature are chosen so as to bring the reaction to completion at a minimum time without the production of unwanted side products. Typically the reaction can be conducted at 0° C. to reflux, preferably 0 to 100° C., more preferably 20-80° C., such 50-70° C., for 6 h to 48 h, preferably 10 h to 36 h, most preferably 12 h to 24 h, such as 20 to 24 h.

Alternatively, a compound of the formula (VI) can be prepared by hydrogenation of a pyrone compound of formula (V′)

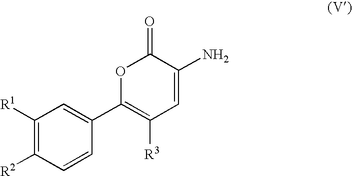

wherein R¹, R² and R³ are as defined for formula (VI), or a salt thereof, to effect ring opening. The hydrogenation preferably takes places under conditions analogous to those described above for compounds (V). In order to incorporate the C(O)R′ group, an anhydride should be employed either simultaneously or subsequently. thus, leading to the, preferably in situ, protection of the amine group. Preferably, the hydrogenation is carried out in the presence of an anhydride. Specifically, the hydrogenation can be conducted with palladium/C in 2-butanol and Boc-anhydride.

Compounds of formula (VI) are prepared from species (V) under amide hydrolysis reactions conditions well known to the person skilled in the art. The hydrolysis of the amide is conducted preferably under acidic conditions, for example, by using 6 M HCl, preferably at elevated temperatures such as 60° C.

The planarity of the substituted pyrone compounds (V) and (V′) enables hydrogenation of the pyrone ring to take place from one face, affording a lactone and defining the relative stereochemistry of the three stereogenic centres of N-substituted 3-amine (C2), 5-alkyl (C4) and 6-aryl (C5). The aryl substituted lactone is benzylic and allows ring opening via hydrogenolysis. The stereochemistry at C5 in the lactone is lost. The catalytic reduction of the pyrone ring to the lactone defines the stereochemistry of the N-substituted 3-amine (C2) and 5-alkyl (C4) and leads to a reduction in the number of possible stereoisomers of the 2-amino-4-alkyl-5-arylpentanoic acid (amino acid) derivatives from four (2S,4S; 2S,4R; 2R,4R and 2R,4S) to two (2S,4S and 2R,4R).

If the compound according to formula (VI) should have a certain stereochemistry, i.e. if it should be present as a single diastereomer, the obtained racemic product can be subjected to optical resolution using methods well known to the person skilled in the art, see e.g. Jacques, J; Collet, A. and Wilen, S. H. (1991) ‘Enantiomers, Racemates and Resolutions’ Reprint, Krieger Publishing Company, Florida ISBN 0-89464-618-4. Most preferably the compound according to formula (VI) is obtained as the (2S,4S) isomer:

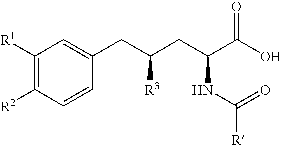

In one embodiment, resolution of compound (VI) is accomplished via enzymatic resolution. Specifically, hydrolysis of the amide under basic conditions (for example in aqueous LiOH) is followed by enantioselective amine acylation by the use of pig kidney acylase. If the (2R, 4R) isomer is selectively acylated over the (2S, 4S) isomer, the free amine of this isomer can be later converted into species (VI) via subsequent protecting group chemistry.

The compound of formula (VI) is a key intermediate in the synthesis of pharmaceutically active substances, preferably renin inhibitors such as aliskiren. Therefore in one embodiment, the present invention also relates to the use of a compound of formula (VI) for the preparation of pharmaceutically active substances, preferably renin inhibitors such as aliskiren.

Although it is possible to employ the pyrone compound of formula (V) in any degree of purity and directly as synthesized, it is preferred to use it as a purified product. This ensures that the compound of formula (VI) is obtained in good yield and purity. The use of the crude pyrone product could led to the formation of unwanted under-reduced lactone, hydrolysed product (from the reaction of the saturated lactone by-product and water from the catalyst reagent) and ester formation (from the reaction of alcoholic solvent and racemate product).

The pyrone itself is a key intermediate in the preparation of the N-substituted 2-amino-4-alkyl-5-arylpentanoic acid and, thus, the synthesis of pharmaceutically active substances, preferably renin inhibitors such as aliskiren. Therefore in one embodiment, the present invention also relates to a compound of formula (V):

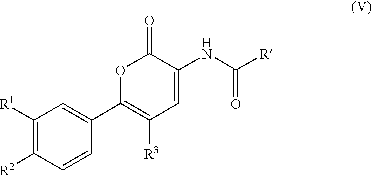

wherein
R¹ is hydrogen, halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof.

In a preferred embodiment, R¹ is hydrogen, hydroxyl, C₁₋₆alkoxy-C₁₋₆alkyloxy or C₁₋₆alkoxy-C₁₋₆alkyl, more preferably C₁₋₄alkoxy-C₁₋₄alkyloxy, most preferably methoxypropoxy.

In a preferred embodiment, R² is hydrogen, hydroxyl or C₁₋₄alkoxy, more preferably C₁₋₄alkoxy, most preferably methoxy.

In a preferred embodiment, R³ is C₁₋₇alkyl, preferably branched C₃₋₆alkyl, most preferably isopropyl.

In a preferred embodiment, R′ is C₁₋₇alkyl or phenyl whereby phenyl can be mono- or di-substituted, preferably C₁₋₆alkyl or phenyl, most preferably methyl or phenyl.

Most preferably, the compound of formula (V) has the following structure:

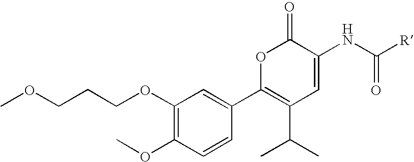

In another embodiment, the present invention relates to a compound of formula (V′):

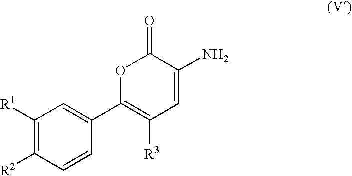

wherein
R¹, R² and R³ are as defined for (V).

In a preferred embodiment, R¹ is hydrogen, hydroxyl, C₁₋₆alkoxy-C₁₋₆alkyloxy or C₁₋₆alkoxy-C₁₋₆alkyl, more preferably C₁₋₄alkoxy-C₁₋₄alkyloxy, most preferably methoxypropoxy. In a preferred embodiment, R² is hydrogen, hydroxyl or C₁₋₄alkoxy, more preferably C₁₋₄alkoxy, most preferably methoxy.

In a preferred embodiment, R³ is C₁₋₇alkyl, preferably branched C₃₋₆alkyl, most preferably isopropyl.

Most preferably, the compound of formula (V′) has the following structure:

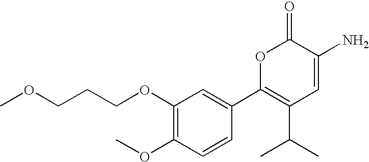

The present inventors have found convenient methods of preparing the key intermediate of the formula (V) as will be described in detail below. Any of the reaction steps either alone or in a suitable combination may be employed to yield the compound of the formula (V). Moreover, any of the following reaction steps either alone or in a suitable combination may be employed in the synthesis of a renin inhibitor, such as aliskiren.

Thus, in a second aspect, the present invention relates to a method for preparing a compound of formula (V) as described above, said method comprising reacting an enamine compound of formula (III)

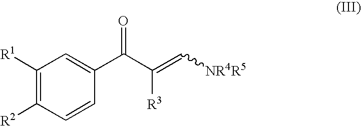

wherein R¹, R² and R³ are as defined for a compound of formula (V), R⁴ and R⁵ are independently C₁₋₆alkyl; preferably methyl or ethyl; or a salt thereof, with an amido glycine derivative of formula (IV) or (IV′) or a tautomer of (IV′)

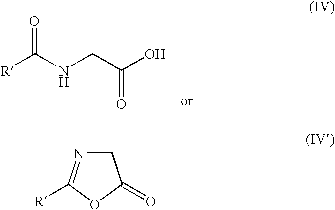

wherein R′ is as defined for a compound of formula (V); or a salt thereof to effect the ring closure to form a pyrone moiety. This process step as such, as well as the compound of formula (III), also form embodiments of the invention.

A tautomer of a compound of formula (IV′) is typically the enol tautomer of formula (IV″). The enol (IV″) and keto (IV′) tautomers are species in equilibrium thus, for sake of convenience and simplicity, it is referred hereinafter only to a compound of formula (IV′) with the intention to embrace both (IV′) and its tautomer (IV″) by this notion.

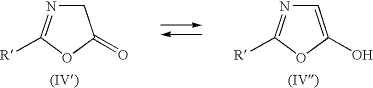

Compounds of the formula (V) can be obtained by the use of the above reaction by methods well known in the art, in particular by following the procedures for preparing pyranones, as disclosed for example; in Renata Toplak, Jurij Svete and Branko Stanovnik, J. Heterocyclic Chem., 1999, 36, 225-235, where the synthesis of 5,6-disubstituted-3-(benzyloxycarbonyl)amino-2H-pyran-2-ones and other heterocycles is detailed; or in Jurij Svete, Zvonko Cadez, Branko Stanovnik and Miha Tisler; Synthesis, 1990, 1, 70-72, where the synthesis of 3-benzoylamino-2H-pyran-2-ones is disclosed.

The reaction to obtain the pyrone moiety preferably takes place under conditions so as to keep the other functionalities on the molecule intact. The conversion of compounds (III) into compounds (V) by reaction with amido glycine derivatives (IV) typically takes place in the presence of an acid anhydride, preferably a low boiling acid anhydride such as one having a boiling point in the rage of 20 to 200° C. Preferred examples include acetic anhydride, propionic anhydride, isobutyric anhydride, n-butyric anhydride and trimethylacetic anhydride, more preferably acetic anhydride. The acid anhydride may be used stoichiometrically or as the solvent (neat), preferably 2 to 200 equivalents, more preferably 2 to 10 equivalents are used. The reaction of compounds (III) with amido glycine derivatives (IV) is usually conducted under an inert atmosphere such as nitrogen or argon. The reaction can take place in an inert solvent, more preferably in tetrahydrofuran, dioxane, benzene, chlorobenzene, toluene, phenylethane, xylenes, most preferably toluene. The reaction time and the temperature are chosen so as to bring the reaction to completion at a minimum time without the production of unwanted side products. Typically the reaction can be conducted at 0° C. to reflux, preferably 20 to 200° C., more preferably 50 to 180° C., such as 100 to 140° C., for 10 min to 3 h, preferably 20 min to 2 h, most preferably 30 min to 50 min, such as 40 min.

The amido glycine derivative of formula (IV) can be used in an amount of 0.9 to 10 equivalent, preferably 1.0 to 1.5 equivalent, such as 1.1 equivalent. Such amido glycine derivatives can be purchased conveniently from suppliers such as Aldrich, Fluka or Acros, or can be obtained by simple peptide chemistry on the glycine amine. The amido glycine derivatives of formula (IV) used in the conversion can be chosen from any suitable amido glycine derivative wherein preferred embodiments of R′ are as set forth for compound (V) above. Most preferably the amido glycine derivative of formula (IV) is hippuric acid or N-acetylglycine.

Alternatively, the conversion of compounds (III) into compounds (V) can be accomplished by reaction with amido glycine derivatives (IV′). The conversion of compounds (III) into compounds (V) by reaction with amido glycine derivatives (IV′) is usually conducted under an inert atmosphere such as nitrogen or argon. The reaction can take place in an inert solvent, more preferably in tetrahydrofuran, dioxane, benzene, chlorobenzene, toluene, phenylethane, xylenes, most preferably toluene. The reaction time and the temperature are chosen so as to bring the reaction to completion at a minimum time without the production of unwanted side products. Typically the reaction can be conducted at 0° C. to reflux, preferably 20 to 200° C., more preferably 50 to 180° C., such as 100 to 140° C., for 10 min to 3 h, preferably 20 min to 2 h, most preferably 40 min to 1 h, such as 1 h.

The amido glycine derivative of formula (IV′) above can be used in an amount of 0.9 to 10 equivalent, preferably 1.0 to 1.5 equivalent, such as 1.1 equivalent. The amido glycine derivatives of formula (IV′) used in the conversion can be chosen from any suitable amido glycine derivative wherein preferred embodiments of R′ are as set forth for compound (V) above.

Typically, amido glycine derivatives of formula (IV′) can be formed from compounds of formula (IV) in the presence of an acid anhydride, preferably a low boiling acid anhydride such as one having a boiling point in the rage of 20 to 200° C. and in the presence of a mild base. Preferred examples of acid anhydrides include acetic anhydride, propionic anhydride, isobutyric anhydride, n-butyric anhydride and trimethylacetic anhydride, more preferably acetic anhydride. The acid anhydride may be used stoichiometrically or as the solvent (neat), preferably 2 to 200 equivalents, more preferably 2 to 10 equivalents are used. Preferred examples of bases include triethylamine, N,N-diisopropylethylamine, N,N-diethylmethylamine, N,N-dimethylethylamine, most preferably triethylamine. Typically the reaction can be conducted at 0° C. to 100° C., preferably 0 to 50° C., more preferably 10 to 30° C., such as 20 to 30° C., for 10 min to 3 h, preferably 20 min to 2 h, most preferably 30 min to 50 min, such as 30 min. Most preferably the amido glycine derivative of formula (IV′) is 2-methyl-4H-oxazol-5-one, which is derived from N-acetylglycine

The product (V) can be used as it is for further conversion(s) but is preferably purified. It can be preferably isolated by trituration in an appropriate solvent such as an alcohol or a mixture of an alcohol and hydrocarbons, such as isopropanol and isopropanol/heptanes.

The enamine itself is a key intermediate in the preparation of the N-substituted 2-amino-4-alkyl-5-arylpentanoic acid and, thus, the synthesis of pharmaceutically active substances, preferably renin inhibitors such as aliskiren. Therefore in one embodiment, the present invention also relates to a compound of formula (III):

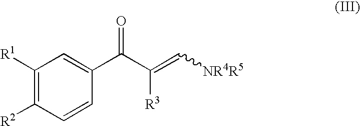

wherein
R¹ is hydrogen, halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R⁴ and R⁵ are independently C₁₋₆alkyl; or a salt thereof.

Preferred embodiments for R¹, R², R³ and R′ are as defined for the compound of formula (V). In a preferred embodiment R⁴ and R⁵ are independently methyl, ethyl, isopropyl, n-propyl or n-butyl, more preferably methyl or ethyl, most preferably methyl. Preferably R⁴ and R⁵ are the same.

Most preferably, the compound of formula (III) has the following structure:

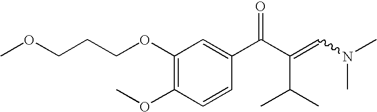

The present inventors have found convenient methods of preparing the key intermediate of the formula (III) as will be described in detail below. This reaction step either alone or in a suitable combination may be employed in the synthesis of a renin inhibitor, such as aliskiren.

Thus, in a third aspect, the present invention relates to a method for preparing a compound of formula (III) as described above, said method comprising reacting an aryl ketone of formula (I)

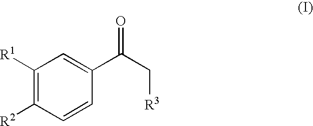

wherein R¹, R² and R³ are as defined for a compound of formula (V), or a salt thereof, with an amine of formula (II)

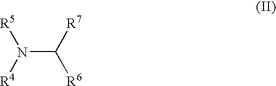

wherein R⁴ and R⁵ are as defined for a compound of formula (III); R⁶ and R⁷ are independently O—C₁₋₆alkyl or NR⁴R⁵, wherein R⁴ and R⁵ are independently as defined for a compound of formula (III); or a salt thereof, to form an enamine moiety. This process step as such also forms an embodiment of the invention. R⁴ in each occurrence can be the same or different. R⁵ in each occurrence can be the same or different. T

Compounds of the formula (III) can be obtained by use of the above reaction by methods well known in the art, in particular by following the procedures for preparing enamines using appropriate ketones. Exemplary methods include those described in Jurij Svete, Zvonko Cadez, Branko Stanovnik and Miha Tisler, Synthesis, 1990, 1, 70-72, where the synthesis of 3-benzoylamino-2H-pyran-2-ones is disclosed; in Harry Wasserman and Jeffrey Ives, J. Org. Chem., 1985, 50, 3573-3580 where the reaction of t-butoxybis(dimethylamino)methane with ketones to form the enamino ketone and subsequent reaction to α-diketones is described; in John Gupton, Keith Krumpe, Bruce Bumham, Kate Dwornik et al, Tetrahedron, 1998, 54, 5075-5088, where the synthesis of enamino carbonyl derivatives of alkylphenones using N,N-dimethylformamidedimethylacetal (DMFDMA) and conversion to a O-chloroenal followed by condensation with ethyl N-substituted glycinate to afford substituted pyrroles is described; in J. Org. Chem., 1978, 43, 21, 4248-4250, where the reaction of DMFDMA with aryl derivatives of acetophenone reagents is disclosed; or in Tetrahedron, 1994, 50, 7, 2255-2264 where the reaction of DMFDMA with simple aryl ketone reagents is described.

The reaction to obtain the enamine moiety preferably takes place under conditions so as to keep the other functionalities on the molecule intact. The reaction is usually conducted under an inert atmosphere such as nitrogen or argon. The reaction can take place neat or in any inert solvent, preferably in an aprotic solvent such as halogenated hydrocarbons, such as methylene chloride; ethers, such as THF, TBME, or dioxane; or aromatic solvents such as benzene, chlorobenzene, toluene, phenylethane, xylenes. Preferably the solvent is toluene. The reaction time and the temperature are chosen so as to bring the reaction to completion at a minimum time without the production of unwanted side products. Typically the reaction can be conducted at 0° C. to reflux, preferably 20 to 200° C., more preferably 60 to 130° C., such as 80 to 110° C., for 6 h to 48 h, preferably 10 h to 36 h, most preferably 12 h to 24 h, such as 20 to 24 h.

In one embodiment, when reacting ketones of formula (II) with amines of formula (I) wherein R⁶ and R⁷ are independently NR⁴R⁵ and, wherein R⁴ and R⁵ are C₁₋₆alkyl, preferably both methyl or ethyl, the reaction takes preferably place in the presence of a base. Most preferably the amine of formula (I) is tris(dimethylamino)methane. The preferred base used is this embodiment is triethylamine, preferably 10 to 50 mol %, more preferably 10 mol %.

In another embodiment, when reacting ketones of formula (II) with amines of formula (I) wherein R⁴ and R⁵ are independently C₁₋₆alkyl, preferably both methyl or ethyl; R⁶ and R⁷ are both O—C₁₋₆alkyl, the reaction also takes place in the presence of a base. Most preferably the amine of formula (I) is dimethylformamidedimethylacetal. The preferred base used is this embodiment is LDA, preferably 2 equivalents.

In another embodiment, when reacting ketones of formula (II) with amines of formula (I) wherein R⁶ is O—C₁₋₆alkyl and R⁷ is NR⁴R⁵ and, wherein R⁴ and R⁵ are C₁₋₆alkyl, preferably both methyl or ethyl, the reaction takes preferably place in the absence of a base. Most preferably the amine of formula (I) is Bredereck's reagent {tert-butoxybis(dimethylamino) methane}

The product (III) can be used as it is for further conversion(s) or can be purified by usual means. Preferably, the compound (III) is used as it is.

The amine of formula (II) used in the conversion can be chosen from any suitable amine falling under the above definition wherein preferred embodiments of R⁴ and R⁵ are as set forth for compound (III) above.

In a preferred embodiment R⁶ is NR⁴R⁵ wherein the preferred definitions for R⁴ and R⁵ are independently the same as set forth for compound (III) above. In another preferred embodiment R⁶ is or O—C₁₋₄alkyl such as O-methyl, O-ethyl, O-isopropyl, O-n-propyl, O-tert-butyl or O-n-butyl, more preferably O-methyl or O-tert-butyl.

In a preferred embodiment R⁷ is NR⁴R⁵ wherein the preferred definitions for R⁴ and R⁵ are independently the same as set forth for compound (III) above. In another preferred embodiment R⁷ is or O—C₁₋₄alkyl such as O-methyl, O-ethyl, O-isopropyl, O-n-propyl, O-tert-butyl or O-n-butyl, more preferably O-methyl or O-tert-butyl.

R⁶ and R⁷ can be the same or can be different. When they are different, it is preferred that one is NR⁴R⁵ and the other is O—C₁₋₆alkyl.

Preferred examples of the amine of formula (II) include Bredereck's reagent {tert-butoxybis(dimethylamino)methane}, methoxybis(dimethylamino)methane, tris(dimethylamino)methane and dimethylformamidedimethylacetal. In one embodiment the amine of formula (II) is most preferably Bredereck's reagent. In another embodiment, the amine of formula (II) is most preferably tris(dimethylamino)methane. In still another embodiment, the amine of formula (II) is most preferably N,N-dimethylformamidedimethylacetal (DMFDMA). The amine of formula (II) can be used in an amount of 1.0 to 10 equivalents, preferably 1.5 to 5 equivalents, such as 3 equivalents. Additional amounts of the reagent can be added such as 1, 2 or 3 equivalents to increase the conversion. The amine can be purchased conveniently from suppliers such as Aldrich, Fluka or Acros, or can be obtained by following the procedures as outlined for example in J. Org. Chem., 1985, 50, 3573-3580.

Alternatively, a compound of formula (V) may be prepared from compounds of formula (I) and (II) without isolation. Therefore, in a fourth embodiment, the present invention relates to a method for preparing a compound of formula (V)

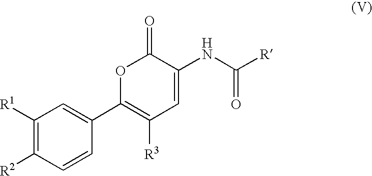

wherein R¹ is hydrogen, halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl, preferably C₁₋₆alkyl or phenyl; or a salt thereof, said method comprising:
a) reacting an aryl ketone of formula (I)

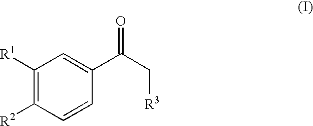

wherein R¹, R² and R³ are as defined for a compound of formula (V), or a salt thereof, with an amine of formula (II)

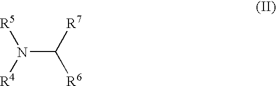

wherein R⁴ and R⁵ are independently C₁₋₆alkyl, preferably both methyl or ethyl; R⁶ and R⁷ are independently NR⁴R⁵ or O—C₁₋₆alkyl, or a salt thereof;
b) followed by reaction with an amido glycine derivative of formula (IV) or (IV′) or a tautomer of (IV′)

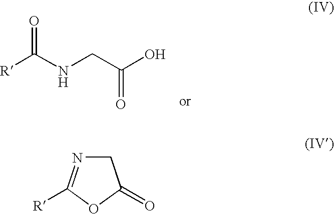

wherein R′ is as defined for a compound of formula (V)

Compounds of the formula (I) can be obtained commercially, e.g. isovalerophenone can be purchased from Fluka. Alternatively, compounds of the formula (I) can be prepared by methods well known in the art, in particular by following the procedures outlined below in Scheme 1 using appropriate commercially available aryl aldehydes.

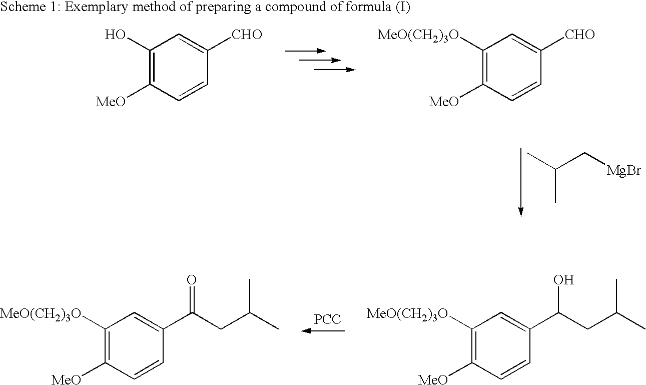

Exemplary methods include those described in Helv. Chim. Act., 2003, 86, 8, 2003 and the final oxidation step is described e.g. in J. Org. Chem., 1995, 60, 2267-2270.

The complete synthesis as encompassed by the present invention is detailed in Scheme 2.

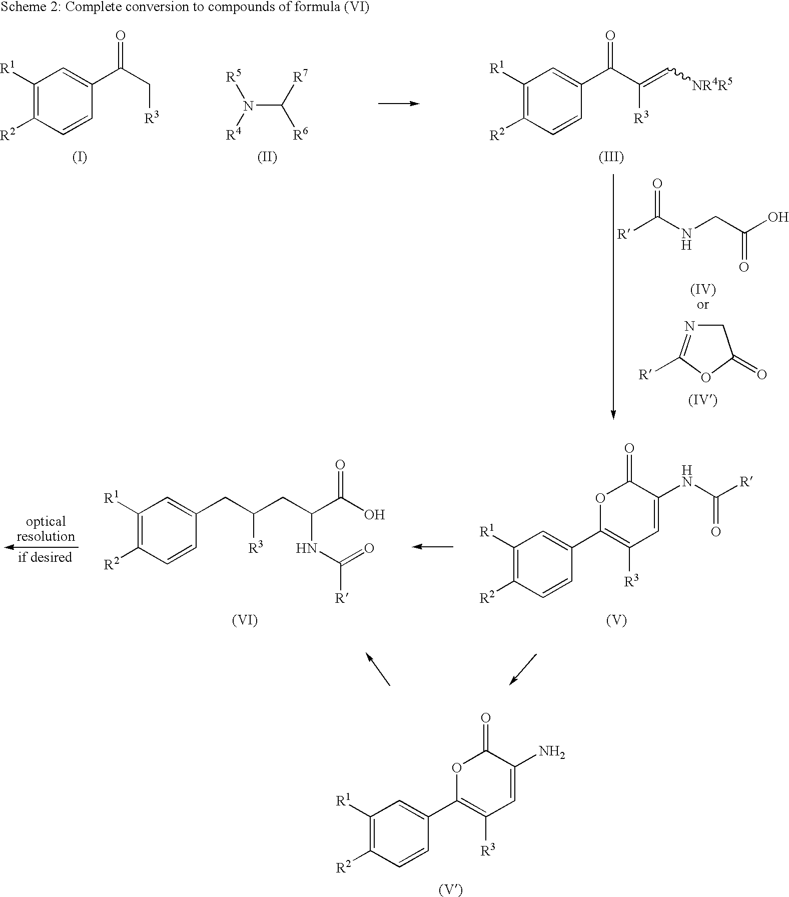

Each conversion as indicated by an arrow can be conducted as a single step. Alternatively, the complete conversion starting from compound (I) can be conducted completely or partially as a one-pot synthesis without further purification of the product. Preferably the conversion from compound (I) to product (V) is conducted in a one-pot synthesis. Preferably the compound (V) is isolated and is preferably further purified before conducting the conversion to obtain compound (VI).

Each of the above mentioned method steps can be used individually in a method to prepare renin inhibitors such as aliskiren. Preferably the steps are used in combination of one or more, most preferably all, to prepare renin inhibitors such as aliskiren.

These synthetic steps show that it is possible to prepare compounds of the formula (VI) which have been found to be central intermediates to a number of possible synthetic routes especially for the synthetic of renin inhibitors such as aliskiren, in an efficient and economic manner using less synthesis steps than previously reported, by proceeding via the important intermediates (III) and (V). Therefore, these compounds of the formulas (III) and (V), or salts thereof, as well as their syntheses form also very highly preferred embodiments of this invention.

One method to prepare renin inhibitors such as aliskiren from compounds of formula (VI) is disclosed in PCT application EP2005/009347 (WO 2006/024501) where these intermediates are depicted as compounds of formula (VII).

Listed below are definitions of various terms used to describe the novel intermediates and synthesis steps of the present invention. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo; where halo is mentioned, this can mean that one or more (e.g. up to three) halogen atoms are present, e.g. in halo-C₁-C₇-alkyl, such as trifluoromethyl, 2,2-difluoroethyl or 2,2,2-trifluoroethyl.

Alkyl preferably has up to 20 carbon atom and is more preferably C₁-C₇-alkyl. Alkyl is straight-chained or branched (one or, if desired and possible, more times). Very preferred is methyl.

Halogenalkyl may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

Branched alkyl preferably comprises 3 to 6 C atoms. Examples are i-propyl, i- and t-butyl, and branched isomers of pentyl and hexyl.

Cycloalkyl preferably comprises 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

Alkenyl may be linear or branched alkyl containing a double bond and comprising preferably 2 to 12 C atoms, 2 to 8 C atoms being especially preferred. Particularly preferred is a linear C₂₋₄alkenyl. Some examples of alkyl groups are ethyl and the isomers of propyl, butyl, pentyl, hexyl, heptyl, octyl, nonyl, decyl, undecyl, dodecyl, tetradecyl, hexadecyl, octacyl and eicosyl, each of which containing a double bond. Especially preferred is allyl.

Alkylamino and dialkylamino may be linear or branched. Some examples are methylamino, dimethylamino, ethylamino, and diethylamino.

Alkoxy-alkyloxy may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

Alkoxyalkyl may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2-ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

Alkoxy may be linear or branched and preferably comprise 1 to 4 C atoms. Examples are methoxy, ethoxy, n- and i-propyloxy, n-, i- and t-butyloxy, pentyloxy and hexyloxy.

Unsubstituted or substituted aryl is preferably a mono- or polycyclic, especially monocyclic, bicyclic or tricyclic aryl moiety with 6 to 22 carbon atoms, especially phenyl (very preferred), naphthyl (very preferred), indenyl, fluorenyl, acenapthylenyl, phenylenyl or phenanthryl, and is unsubstituted or substituted by one or more, especially one to three, moieties, preferably independently selected from the group consisting of C₁-C₇-alkyl, C₁-C₇-alkenyl, C₁-C₇-alkynyl, halo-C₁-C₇-alkyl, such as trifluoromethyl, halo, especially fluoro, chloro, bromo or iodo, hydroxy, C₁-C₇-alkoxy, phenyloxy, naphthyloxy, phenyl- or naphthyl-C₁-C₇-alkoxy, C₁-C₇-alka-noyloxy, phenyl- or naphthyl-C₁-C₇-alkanoyloxy, amino, mono- or di-(C₁-C₇-alkyl, phenyl, naphthyl, phenyl-C₁-C₇-alkyl, naphthyl-C₁-C₇-alkyl, C₁-C₇-alkanoyl and/or phenyl- or naphthyl-C₁-C₇-alkanoyl)-amino, carboxy, C₁-C₇-alkoxycarbonyl, phenoxycarbonyl, naphthyloxycarbonyl, phenyl-C₁-C₇-alkyloxycarbonyl, naphthyl-C₁-C₇-alkoxycarbonyl, carbamoyl, N-mono- or N,N-di-(C₁-C₇-alkyl, phenyl, naphthyl, phenyl-C₁-C₇-alkyl and/or naphthyl-C₁-C₇-alkyl)-aminocarbonyl, cyano, sulfo, sulfamoyl, N-mono- or N,N-di-(C₁-C₇-alkyl, phenyl, naphthyl, phenyl-C₁-C₇-alkyl and/or naphthyl-C₁-C₇-alkyl)-aminosulfonyl and nitro.

Salts are especially the pharmaceutically acceptable salts of compounds of formula VI or generally salts of any of the intermediates mentioned herein, where salts are not excluded for chemical reasons the skilled person will readily understand. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as base addition salts, preferably with organic or inorganic bases, from compounds of formula VI or any of the intermediates mentioned herein with an acidic carboxy group, especially the pharmaceutically acceptable salts. Suitable metal ions from inorganic bases are, for example, alkaline or alkaline earth metals, such as sodium, potassium, magnesium or calcium salts. Suitable organic bases are, for example, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

In the presence of positively charged radicals, such as amino, salts may also be formed with acids. Such salts are formed, for example, as acid addition salts, preferably with organic or inorganic acids. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

When a basic group and an acid group are present in the same molecule, a compound of formula VI or any of the intermediates mentioned herein may also form internal salts.

For isolation or purification purposes of compounds of the formula VI or in general for any of the intermediates mentioned herein it is also possible to use pharmaceutically unacceptable salts, for example picrates or perchlorates. For therapeutic use, only pharmaceutically acceptable salts or free compounds of the formula VI are employed (where applicable comprised in pharmaceutical preparations), and these are therefore preferred at least in the case of compounds of the formula VI.

In view of the close relationship between the compounds and intermediates in free form and in the form of their salts, including those salts that can be used as intermediates, for example in the purification or identification of the compounds or salts thereof, any reference to “compounds”, “starting materials” and “intermediates” hereinbefore and hereinafter, especially to the compound(s) of the formula VI, is to be understood as referring also to one or more salts thereof or a mixture of a corresponding free compound, intermediate or starting material and one or more salts thereof, each of which is intended to include also any solvate, metabolic precursor such as ester or amide of the compound of formula VI, or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is not intended to exclude the plural, but only preferably means “one”.

Starting materials are especially the compounds of the formula I, II and/or IV mentioned herein, intermediates are especially compounds of the formula III and/or V.

The invention relates also to methods of synthesis of the intermediates of the formula III and V mentioned above from their respective precursors as mentioned above, including methods with the single steps of a sequence leading to a compound of the formula VI, more than one or all steps of said synthesis, and/or leading to pharmaceutically active substances, especially renin inhibitors, most preferably aliskiren, including methods with the single steps of a sequence leading to a compound of the formula VI, more than one or all steps of said synthesis, and/or their use in the synthesis of pharmaceutically active compounds, such as renin inhibitors, especially aliskiren.

### General Process Conditions

The following, in accordance with the knowledge of a person skilled in the art about possible limitations in the case of single reactions, applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula VI is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T.

W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/l, Georg Thieme Verlag, Stuttgart 1974, in H.-D. Jakubke and H. Jeschkeit, “Aminosacuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974, all of which are incorporated herein by reference. A characteristic of protecting groups is that they can be removed readily (i.e. without the occurrence of undesired secondary reactions) for example by solvolysis, reduction, photolysis or alternatively under physiological conditions (e.g. by enzymatic cleavage). Different protecting groups can be selected so that they can be removed selectively at different steps while other protecting groups remain intact. The corresponding alternatives can be selected readily by the person skilled in the art from those given in the standard reference works mentioned above or the description or the Examples given herein.

All the above-mentioned process steps can be carried out under reaction conditions that are known per se, preferably those mentioned specifically, in the absence or, customarily, in the presence of solvents or diluents, preferably solvents or diluents that are inert towards the re-agents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100 C to about 190 C, preferably from approximately −80 C to approximately 150 C, for example at from −80 to −60 C, at room temperature, at from −20 to 40 C or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning. Where required or desired, water-free or absolute solvents can be used.

Where required, the working-up of reaction mixtures, especially in order to isolate desired compounds or intermediates, follows customary procedures and steps, e.g. selected from the group comprising but not limited to extraction, neutralization, crystallization, chromatography, evaporation, drying, filtration, centrifugation and the like.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula VI described as being preferred. Special preference is given to reaction conditions that are identical or analogous to those mentioned in the Examples. The invention relates also to novel starting compounds and intermediates described herein, especially those leading to compounds mentioned as preferred herein.

The invention especially relates to any of the methods described hereinbefore and hereinafter that leads to aliskiren, or a pharmaceutically acceptable salt thereof.

The following Examples serve to illustrate the invention without limiting the scope thereof, while they on the other hand represent preferred embodiments of the reaction steps, inter-mediates and/or the process of manufacture of aliskiren, or salts thereof.

### Examples

### Synthesis of racemic (R,R)-(S,S)-2-Benzoylamino-4-benzyl-5-methylhexanoic acid (IVa)

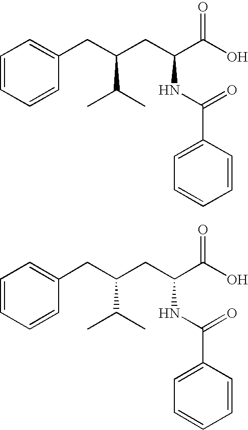

### Synthesis of intermediate N-(5-Isopropyl-2-oxo-6-phenyl-2H-pyran-3-yl)benzamide (Va)

Under an inert atmosphere (N₂), tert-butoxybis(dimethylamino)methane (IIa) (18.5 mL, 15.6 g, 90 mmol) is charged to a stirred mixture of isovalerophenone (Ia) (7.5 mL, 7.25 g, 45 mmol) in anhydrous toluene (75 mL) at room temperature. The resulting mixture is stirred at reflux for 20 h. The volatiles are removed under reduced pressure to yield a crude orange oil characterized as a mixture of desired enamine intermediate (IIIa) and starting isovalerophenone (Ia).

Isovalerophenone (Ia): ¹H NMR (CDCl₃) 1.00 (d, 6H, J=6.6 Hz, CH(CH₃)₂), 2.30 (sept, 1H, J=6.6 Hz, CH(CH₃)₂), 2.84 (d, 2H, J=7.1 Hz, CH₂), 7.43-7.45 (m, 2H, PhCH), 7.54-7.57 (m, 1H, PhCH), 7.94-7.96 (m, 2H, PhCH).

2-Dimethylaminomethylene-3-methyl-1-phenylbutan-1-one (IIIa): ¹H NMR (CDCl₃) 1.37 (d, 6H, J=6.8 Hz, CH(CH₃)₂), 2.98 (s, 6H, N(CH₃)₂), 3.12 (sept, 1H, J=6.8 Hz, CH(CH₃)₂), 6.65 (s, 1H, CHN(CH₃)₂), 7.33-7.48 (m, 5H, PhCH).

To the crude residue is added hippuric acid (IVa) (8.8 g, 49 mmol) and acetic anhydride (75 mL) at room temperature and the resulting mixture is stirred at reflux for 40 min. The volatiles are removed under reduced pressure and the resulting crude product is triturated in cold (0-4° C.) isopropanol (40 mL). The precipitate is collected by filtration, washed with small portion of cold (04° C.) isopropanol (3×4 mL) and dried by suction under air at room temperature to yield the desired pyrone (Va) as a crystalline beige solid.

### N-(5-Isopropyl-2-oxo-6-phenyl-2H-pyran-3-yl)benzamide (Va)

¹H NMR (CDCl₃) 1.24 (d, 6H, J=6.8 Hz, CH(CH₃)₂), 3.08 (sept, 1H, J=6.8 Hz, CH(CH₃)₂), 7.45-7.59 (m, 8H, PhCH), 7.92-7.94 (m, 2H, PhCH), 8.63 (s, 1H, pyroneCH), 8.78 (s, 1H, NH).

¹³C NMR (CDCl₃) 22.62 (CH(CH₃)₂), 27.71 (CH(CH₃)₂), 123.95, 127.06, 128.39, 128.84, 128.87, 129.54 and 132.40(PhCH and pyroneCH), 123.13, 124.61, 132.03, 133.58 and 150.12 (PhC and pyroneC), 159.67 and 166.10 (CO).

### Synthesis of racemic (R,R)-(S,S)-2-Benzoylamino-4-benzyl-5-methylhexanoic acid (VIa)

A pressure vessel is charged with N-(5-isopropyl-2-oxo-6-phenyl-2H-pyran-3-yl)benzamide (Va) (0.5 g, 1.5 mmol), palladium hydroxide on charcoal—20%, 60% wet (66 mg, 0.13 wt) and isopropanol (20 mL). The pressure vessel is sealed, degassed by vac-N₂ cycle followed by vac-H₂ cycle, and finally set at the desired H₂ pressure (8 bar). The mixture is then stirred at 60° C. for 20 h. The mixture is filtered over celites, the pad is rinsed with isopropanol and the filtrates are combined and concentrated in vacuum. The crude product is recrystallised from a mixture of EtOAc-heptanes (1:4) at 0-4° C. to yield the desired product (VIa) as a white crystalline solid.

### Racemic (R,R)-(S,S)-2-Benzoylamino-4-benzyl-5-methylhexanoic acid (VIa)

¹H NMR (CD₃OD) 0.87 (dd, 6H, J=6.9 Hz and 12.5 Hz, CH(CH₃)₂), 1.67-1.94 (m, 4H, CH(CH₃)₂, CHCH(CH₃)₂ and NHCHCH₂), 2.53 and 2.77 (m, 2H, Ph-CH₂), 4.80 (m, 1H, NHCH), 7.09-7.22 (m, 5H, PhCH), 7.44-7.55 (m, 3H, PhCH), 7.84-7.86 (m, 2H, PhCH).

¹³C NMR (CD₃OD) 17.51 and 20.45 (CH(CH₃)₂), 29.15 (CH(CH₃)₂), 33.12 (NHCHCH₂), 37.85 (Ph-CH₂), 43.77 (CHCH(CH₃)₂), 52.31 (NHCH), 126.84, 128.53, 129.28, 129.30, 129.59, 130.14 and 132.83 (PhCH), 135.52 and 142.54 (PhC), 170.52 and 176.05 (CO).

### Synthesis of racemic (R,R)-(S,S)-2-Acetylamino-4-[4-methoxy-3-(3-methoxypropoxy)benzyl]-5-methylhexanoic acid (VIb)

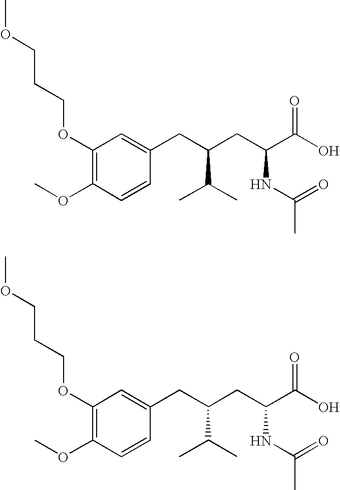

### Synthesis of intermediate N-{5-isopropyl-6-[4-methoxy-3-(3-methoxypropoxy)phenyl]-2-oxo-2H-pyran-3-yl}acetamide (Vb)

Under an inert atmosphere (N₂), tert-butoxybis(dimethylamino)methane (IIa) (50.0 mL, 42.2 g, 242 mmol) is added to a stirred mixture of 1-[4-methoxy-3-(3-methoxy-propoxy)-phenyl]-3-methyl-butan-1-one (Ib) (25.4 g, 91 mmol) in anhydrous toluene (100 mL) at room temperature. The resulting mixture is stirred at reflux for 20 h. The volatiles are removed under reduced pressure to yield a crude orange oil characterised as a mixture of the desired enamine intermediate (IIIb) and starting 1-[4-methoxy-3-(3-methoxy-propoxy)-phenyl]-3-methyl-butan-1-one (Ib).

1-[4-Methoxy-3-(3-methoxnpropoxy)phenyl]-3-methylbutan-1-one (Ib): ¹H NMR (CDCl₃) 0.99 (d, 6H, J=6.6 Hz, CH(CH₃)₂), 2.13 (quint, 2H, J=6.4 Hz, CH₂CH₂O), 2.29 (sept, 1H, J=6.6 Hz, CH(CH₃)₂), 2.78 (d, 2H, J=6.8 Hz, COCH₂), 3.37 (s, 3H, OCH₃), 3.58 (t, 2H, J=6.4 Hz, CH₂O), 3.93 (s, 3H, OCH₃), 4.19 (t, 2H, J=6.6 Hz, CH₂O), 6.89 (d, 1H, J=8.0 Hz, ArCH), 7.57-7.59 (m, 2H, ArCH).

2-Dimethylaminomethylene-1-[4-methoxy-3-(3-methoxypropoxy)phenyl]-3-methylbutan-1-one (IIIb): ¹H NMR (CDCl₃) 1.28 (d, 6H, J=7.1 Hz, CH(CH₃)₂), 2.09 (quint, 2H, J=6.4 Hz, CH₂CH₂O), 2.91 (s, 6H, N(CH₃)₂), 3.05 (sept, 1H, J=6.8 Hz, CH(CH₃)₂), 3.28 (s, 3H, OCH₃), 3.50 (t, 2H, J=6.4 Hz, CH₂O), 3.82 (s, 3H, OCH₃), 4.09 (t, 2H, J=6.6 Hz, CH₂O), 6.62 (s, 1H, CHN(CH₃)₂), 6.72 (d, 1H, J=8.3 Hz, ArCH), 6.97 (m, 1H, ArCH), 7.08 (m, 1H, ArCH).

To the crude residue is added N-acetyl glycine (IVb) (10.4 g, 89 mmol) and acetic anhydride (100 mL) at room temperature and, the resulting mixture is stirred at reflux for 40 min. The volatiles are removed under reduced pressure. The resulting crude product is taken in ethyl acetate (250 mL) and water (200 mL). The organic phase is extracted and washed with water (200 mL), brine (100 mL), dried over MgSO₄ (20 g), filtered and concentrated in vacuum. The crude solid residue is slurried in heptanes (500 mL), filtered and then slurried in a mixture of isopropanol/heptanes (1:4) (185 mL). The suspension is isolated by filtration and the solid is washed with small portions of a mixture of isopropanol/heptanes (1:4) (3×18.5 mL), and then dried by suction under air at room temperature to yield the desired pyrone (Vb) as a beige solid.

### N-{5-Isopropyl-6-[4-methoxy-3-(3-methoxypropoxy)phenyl]-2-oxo-2H-pyran-3-yl}acetamide (Vb)

¹H NMR (CDCl₃) 1.12 (d, 6H, J=6.9 Hz, CH(CH₃)₂), 2.05 (quint, 2H, J=6.4 Hz, CH₂CH₂O), 2.15 (s, 3H, NCOCH₃), 2.99 (sept, 1H, J=6.9 Hz, CH(CH₃)₂), 3.28 (s, 3H, OCH₃), 3.50 (t, 2H, J=6.1 Hz, CH₂O), 3.84 (s, 3H, OCH₃), 4.07 (t, 2H, J=6.6 Hz, CH₂O), 6.84 (d, 1H, J=8.6 Hz, Ar—CH), 6.95-6.97 (m, 2H, Ar—CH), 7.93 (s, 1H, NH), 8.34 (s, 1H, pyroneCH).

¹³C NMR (CDCl₃) 22.66 (CH(CH₃)₂), 24.70 (CH(CH₃)₂), 27.81 (NHCOCH₃), 29.51 (OCH₂CH₂), 56.01 and 58.70 (OCH₃), 66.20 and 69.16 (OCH₂), 110.98, 113.58, 121.95 and 124.16 (Ar—CH and pyroneCH), 122.60, 124.08, 124.63, 148.28, 150.13 and 150.54 (Ar—C and pyroneC), 159.60 and 169.35 (CO).

### Synthesis of racemic (R,R)-(S,S)-2-Acetylamino-4-[4-methoxy-3-(3-methoxypropoxy)benzyl]-5-methylhexanoic acid (VIb)

A pressure vessel is charged with N-5-isopropyl-6-[4-methoxy-3-(3-methoxy-propoxy)-phenyl]-2-oxo-2H-pyran-3-yl-acetamide (Vb) (8.7 g, 22 mmol), palladium on charcoal—5%, 50% wet (5.2 g, 0.6 wt) and n-butanol (200 mL). The pressure vessel is sealed, degassed by vac-N₂ cycle followed by vac-H₂ cycle, and finally set at the desired H₂ pressure (8 bar). The mixture is then stirred at 80° C. for 20 h. The mixture is filtered over celites, the pad is rinsed with isopropanol and the filtrates are combined and concentrated in vacuum to yield the desired product (VIb) as a colourless oil.

### Racemic (R,R)-(S,S)-2-Acetylamino-4-[4-methoxy-3-(3-methoxypropoxy)benzyl]-5-methyl hexanoic acid (VIb)

¹H NMR (CDCl₃) 0.86 (dd, 6H, J=6.8 Hz and 14.0 Hz, CH(CH₃)₂), 1.59-1.66 and 1.76-1.81 (m, 4H, CH(CH₃)₂, CHCH(CH₃)₂ and NHCHCH₂), 1.98 (s, 3H, NCOCH₃), 2.10 (quint, 2H, J=6.4 Hz, CH₂CH₂O), 2.50 (m, 2H, ArCH₂), 3.36 (s, 3H, OCH₃), 3.61 (m, 2H, CH₂O), 3.81 (s, 3H, OCH₃), 4.11 (m, 2H, CH₂O), 4.58 (m, 1H, NHCH), 6.14 (d, 1H, J=8.4 Hz, NH), 6.67 (m, 1H, Ar—CH), 6.73-6.78 (m, 2H, Ar—CH), 8.91 (b, 1H, COOH).

¹³C NMR (CDCl₃) 17.90 and 19.26 (CH(CH₃)₂), 22.84 (NHCOCH₃), 28.16 (CH(CH₃)₂), 29.12 (OCH₂CH₂), 33.05 (NHCHCH₂), 36.08 (ArCH₂), 41.87 (CHCH(CH₃)₂), 50.77 (ArCH₂), 55.94 and 58.44 (OCH₃), 65.85 and 69.42 (OCH₂), 111.56, 114.22 and 121.59 (Ar—CH), 133.47, 147.60 and 147.99 (Ar—C), 170.70 and 175.11 (CO).

### Synthesis of intermediate 2-dimethylaminomethylene-1-[4-methoxy-3-(3-methoxypropoxy) Phenyl]-3-methylbutan-1-one (IIIb) using tris(dimethylamino)methane (IIb)

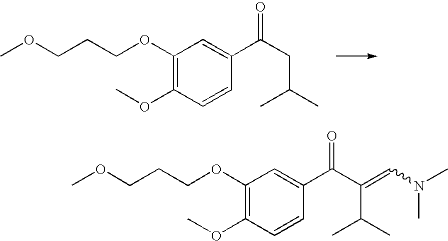

A mixture of 1-[4-Methoxy-3-(3-methoxypropoxy)phenyl]-3-methyl-butan-1-one (Ib) (100 g, 0.358 mol) (Ib), triethylamine (3.6 g, 0.0356 mol) and tris(dimethylamino)methane (IIb) (62 g, 0.4269 mol) in toluene (800 mL) is heated to reflux until >90% conversion is observed.

¹H NMR (D₆-benzene) 1.4 and 1.8 (d, 6H, J=6.85 Hz, (CH₃)₂CH), 2.05 (m, 2H, CH₃OCH₂CH₂CH₂O), 2.3 and 2.35 (s, 6H, (CH₃)₂N), 3.0 and 3.2 (septet, 1H, J=6.85 Hz, (CH₃)₂CH), 3.1 (s, 3H, CH₃OCH₂CH₂CH₂O), 3.5 (m, 5H, CH₃OCH₂CH₂CH₂O and CH₃OAr), 6.2 and 6.8 (s, 1H, CHN(CH₃)₂), 6.6 (dd, 1H, J=8.3, J=3.42, CH_{Ar}), 7.4 and 7.8 (dd, 1H, J=8.3, J=1.95, CH_{Ar}), 7.6 and 7.85 (d, 1H, J=1.95, CHAR).

¹³C NMR (D₆-benzene) 22.5 and 24.2 ((CH₃)₂CH), 27.3 and 32.9 ((CH₃)₂CH), 30.0 (CH₃OCH₂CH₂CH₂O), 43.0 and 43.1 ((CH₃)₂N), 55.3 and 55.5 (CH₃OAr), 58.3 (CH₃OCH₂CH₂CH₂O), 65.9 (CH₃OCH₂CH₂CH₂O), 69.3 (CH₃OCH₂CH₂CH₂O), 114.6 and 117.5 (CCHN(CH₃)₂), 142.8 and 152.4 (CCHN(CH₃)₂), 110.6, 110.8, 112.5, 114.6, 122.4, 123.4, 134.9, 136.4, 148.9, 149.3, 151.6 and 153.1 (Aryl-C).

### Synthesis of N-{5-isopropyl-6-[4-methoxy-3-(3-methoxypropoxy)phenyl]-2-oxo-2H-pyran-3-yl}acetamide (Vb) from 1-[4-methoxy-3-(3-methoxypropoxy)-phenyl]-3-methylbutan-1-one (Ib) using tris(dimethylamino)methane (IIb) and N-acetylglycine (IVb)

1-[4-Methoxy-3-(3-methoxypropoxy)phenyl]-3-methylbutan-1-one (Ib) (2.2 g, 7.9 mmol), toluene (18 mL), triethylamine (0.079 g, 0.8 mmol) and tris(dimethylamino)methane (IIb) (1.36 g, 9.4 mmol) are charged to a vessel and heated to 113° C. in order to achieve reflux. The reaction is held at said temperature until >90% conversion is observed. The reaction solution is cooled to 40° C. and the solvent is removed by vacuum distillation. N-acetylglycine (IVb) (0.91 g, 7.8 mmol), toluene (8.8 mL) and acetic anhydride (4.4 mL) are added to the residue and the resultant mixture is heated to 110° C. Complete consumption of enamine is observed after 1 hour. The reaction is cooled to 30-40° C. and the solvent is removed by vacuum distillation. Ethyl acetate (18 mL) and water (18 mL) are added to the residue and the phases mixed vigorously. The phases are separated and the aqueous phase back extracted with EtOAc (18 mL). The combined organic phases are washed with water (9 mL) and dried over Na₂SO₄. The dried organic solution is concentrated under reduced pressure at 30-40° C. to afford crude N-{5-isopropyl-6-[4-methoxy-3-(3-methoxypropoxy)phenyl]-2-oxo-2H-pyran-3-yl}acetamide (Vb) as an orange solid. The crude product is slurried in 10% v/v IPA/heptanes (8 vol) for 2 hours and then isolated by filtration. The filter cake is washed with 10% v/v IPA/heptanes (4.4 mL) and dried on the filter for 1 hour. The product (Vb) is isolated as an off white solid.

### Synthesis of 2-methyl-4H-oxazol-5-one (IV′a or IV″a)

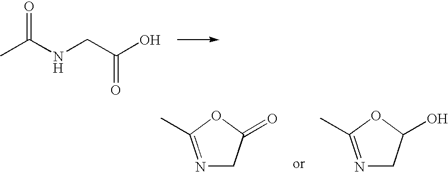

N-acetylglycine (IVb) (54 g, 0.4611 mol), toluene (270 mL) and acetic anhydride (142 g) are charged to an appropriately sized vessel under nitrogen and the temperature adjusted to 20° C. Triethylamine (47 g, 0.4644 mol) is charged over 30 minutes whilst maintaining the temperature at 20-26° C. IPC analysis after 30 minutes shows complete consumption of N-acetylglycine. The reaction product is used as a crude solution without purification.

¹H NMR (D₆-DMSO) 2.3 (s, 3H, CH₃), 4.2 (s, 1H, CH).

### Synthesis of N-{5-isopropyl-6-[4-methoxy-3-(3-methoxypropoxy)phenyl]-2-oxo-2H-pyran-3-yl}acetamide (Vb)

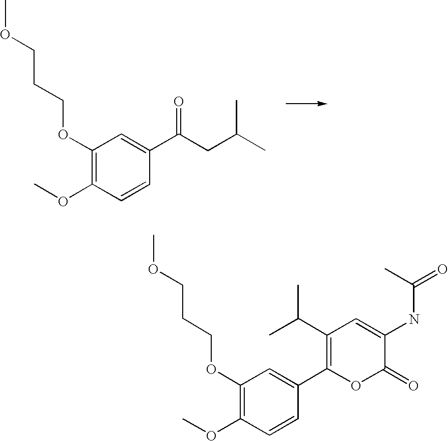

### Method 1: By using 2-dimethylaminomethylene-1-[4-methoxy-3-(3-methoxypropoxy)phenyl]-3-methylbutan-1-one (IIIb) and 2-methyl-4H-oxazol-5-one

1-[4-methoxy-3-(3-methoxypropoxy)phenyl]-3-methylbutan-1-one (Ib) (100 g, 0.358 mol), toluene (800 mL), triethylamine (3.6 g, 0.0356 mol) and tris(dimethylamino)methane (IIb) (62 g, 0.4268 mol) are charged to a vessel and heated to 113° C. in order to achieve reflux. The reaction is held at said temperature until >90% conversion is observed. Whilst maintaining reflux, the solution of 2-methyl-4H-oxazol-5-one prepared above is charged over 1 hour. Complete consumption of enamine is observed after 1 hour. The reaction is cooled to 30-40° C. and the solvent is removed by vacuum distillation. Toluene (1 L) is charged and the solvent is removed by vacuum distillation. Ethyl acetate (600 mL) and water (300 mL) are charged to the residue and the phases mixed vigorously. The phases are separated and the aqueous phase back extracted with ethyl acetate (250 mL). The combined organic phases are washed with water (250 mL) and dried over Na₂SO₄. The dried organic solution is concentrated under reduced pressure at 30-40° C. to afford crude N{5-isopropyl-6-[4-methoxy-3-(3-methoxypropoxy)phenyl]-2-oxo-2H-pyran-3-yl}acetamide (Vb) as an orange solid. The crude product is slurried in 10% v/v IPA/heptanes (8 vol) for 2 hours and then isolated by filtration. The filter cake is washed with 10% v/v IPA/heptanes (3×100 mL) and dried on the filter for 3 hours. The product (Vb) is isolated as an off white solid.

### Method 2: By using 1-[4-methoxy-3-(3-methoxypropoxy)phenyl]-3-methyl-butan-1-one (Ib) and dimethylformamidedimethylacetal (IIc), followed by reaction with N-acetylglycine (IVb)

Lithium diisopropylamide in THF (2.0 M, 35.7 mL, 71.3 mmol) is added slowly to a solution of 1-[4-methoxy-3-(3-methoxypropoxy)phenyl]-3-methylbutan-1-one (Ib) (10 g, 35.7 mmol) in tetrahydrofuran (80 mL) at 0-4° C. Stirring is continued for 1 hour prior to addition of dimethylformamidedimethylacetal (IIc) (8.5 g, 71.3 mmol). The mixture is warmed to 15-25° C. for 1 hour and then is heated to reflux overnight. The volatiles are removed under reduced pressure and diethyl ether (50 mL) is added to the crude residue. The resulting suspension is filtered and the filtrates are concentrated to dryness under reduced pressure. To the resulting crude product is added N-acetylglycine (IVb) (4.2 g, 35.9 mmol) and toluene (48 mL). The solution is cooled to 0-4° C., acetic anhydride (24 mL) is added and the reaction mixture is stirred at reflux temperature for 1 hour. The reaction mixture is concentrated to dryness under vacuum. Water (120 mL) and ethyl acetate (120 mL) are charged. The aqueous layer is further extracted with ethyl acetate (2×120 mL). The combined organic extracts are washed with brine (120 mL), dried over MgSO₄ and concentrated to dryness under reduced pressure. The resulting solid is slurried in heptanes/2-propanol (9:1, 120 mL) and collected by filtration. The filter cake is washed with heptanes/2-propanol (9:1, 12 mL) and dried on the filter to give the desired product (Vb) as a beige powder.

### Method 3: Variation of Method 2

Lithium diisopropylamide in THF (2.0 M, 35.7 mL, 71.3 mmol) is added slowly to a solution of 1-[4-methoxy-3-(3-methoxypropoxy)phenyl]-3-methylbutan-1-one (Ib) (10 g, 35.7 mmol) in ethylene glycol dimethyl ether (80 mL) at 0-4° C. Stirring is continued for 1 hour prior to addition of dimethylformamidedimethylacetal (IIc) (8.5 g, 71.3 mmol). The mixture is warmed to 15-25° C. over 1 hour before heating to reflux temperature overnight. The reaction mixture is cooled to 0-4° C. and the resulting solids are removed by filtration. N-Acetylglycine (Ivb) (4.2 g, 35.9 mmol) is added to the filtrate before cooling to 0-4° C. Acetic anhydride (24 mL) is added and the reaction mixture is heated to 60° C. overnight. The reaction mixture is concentrated to dryness under reduced pressure. Water (120 mL) and ethyl acetate (120 mL) are charged. The aqueous layer is further extracted with ethyl acetate (2×120 mL). The combined organic extracts are washed with brine (120 mL), dried over MgSO₄ and concentrated to dryness under reduced pressure. The resulting solid is slurried in heptanes/2-propanol (9:1, 120 mL) and collected by filtration. The filter cake is washed with heptanes (12 mL) and dried on the filter to give the desired product (Vb) as a beige powder.

### Synthesis of 3-amino-5-isopropyl-6-[4-methoxy-3-(3-methoxypropoxy)phenyl]pyran-2-one

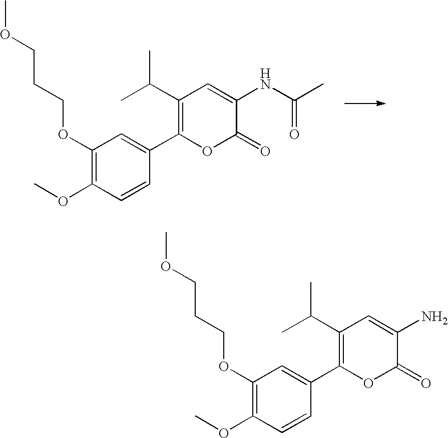

N-{5-isopropyl-6-[4-methoxy-3-(3-methoxypropoxy)phenyl]-2-oxo-2H-pyran-3-yl}acetamide (Vb) (77 g, 1 wt), acetic acid (400 mL) and 6 M hydrochloric acid (400 mL) are charged to a vessel and heated to 60° C. for 2 hours. The solvent is removed by vacuum distillation and the residue azeotropically dried with toluene (4×100 mL). The residue is dissolved in water (200 mL) and the pH adjusted to 7 with 1 M KOH. The product is extracted with toluene (2×400 mL) and then dried over MgSO₄. The solvent is removed by vacuum distillation to give the desired product (V′a) as a viscous oil; ¹H-NMR (CDCl₃) 1.05 (d, 6H, J=6.85, CH(CH₃)₂), 2.05 (quint, 2H, J=6.4 Hz, CH₂CH₂O), 2.95 (septet, 1H, J=6.85 Hz, CH(CH₃)₂), 3.3 (s, 3H, OCH₃), 3.45 (t, 2H, J=6.1 Hz, CH₂O), 3.84 (s, 3H, OCH₃), 4.07 (t, 2H, J=6.6 Hz, CH₂O), 6.4 (s, 1H, pyroneCH), 6.84 (d, 1H, J=8.3 Hz, Ar—CH), 6.95-7.0 (m, 2H, Ar—CH).

### Synthesis of racemic (R,R)-(S,S)-2-tert-butoxycarbonylamino-4-[4-methoxy-3-(3-methoxy propoxy)benzyl]-5-methylhexanoic acid (VIc) from 3-amino-5-isopropyl-6-[4-methoxy-3-(3-methoxypropoxy)phenyl]pyran-2-one (V′b)

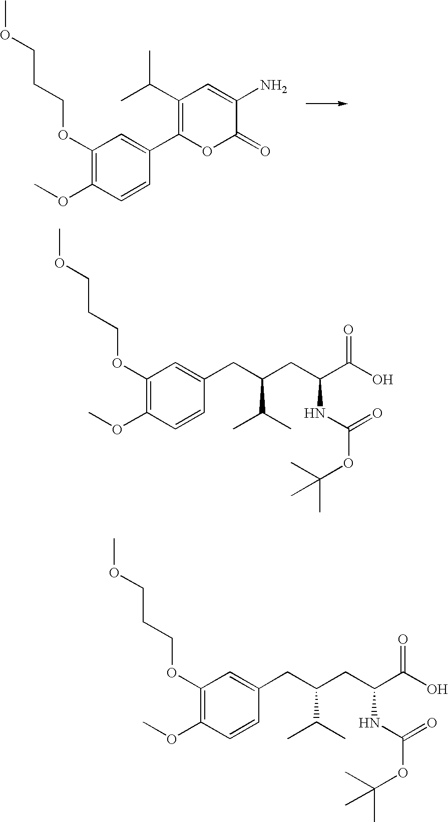

Pd/C (5% wt 50% wet, Degussa type E101, 12 g), 3-amino-5-isopropyl-6-[4-methoxy-3-(3-methoxypropoxy)phenyl]pyran-2-one (V′a) (24 g, 69.1 mmol) in 2-butanol (240 mL) and Boc anhydride (16.8 g, 77 mmol) are charged under nitrogen to a pressure vessel. The mixture is purged 3 times with vacuum/nitrogen, followed by 3 vacuum/hydrogen (at 3 bar) cycles. The reaction mixture is placed under 3 bar of hydrogen and heated to 55-65° C. for 1-2 hours, adjusting the hydrogen pressure continuously to 3 bar. The reaction is then heated to 95-105° C. for 1-2 hours. Upon completion, the reaction mixture is cooled to 15-25° C. and purged 3 times with vacuum/nitrogen. The reaction mixture is then heated to 45-55° C. and filtered through a 1 μm filter membrane. The solids are washed with warm 2-butanol (45-55° C., 2×120 mL). The resultant 2-butanol filtrates are concentrated to ca. 5 volumes to provide a solution of racemic 2-tert-butoxycarbonylamino-4-[4-methoxy-3-(3-methoxypropoxy)benzyl]-5-methylhexanoic acid (VIc) in 2-butanol.

¹H NMR (CDCl₃) 0.9 (m, 6H, CH(CH₃)₂), 1.4 (s, 9H, Boc), 1.6 (dd, 2H, CH₂), 1.7-1.8 (m, 2H, 2×CH), 2.1 (m, 2H, CH₂), 2.4 (m, 2H, ArCH₂), 3.4 (s, 3H, CH₃O), 3.6 (m, 2H, CH₂O), 3.8 (s, 3H, CH₃O), 4.1-4.2 (m, 2H, CH₂O), 4.3 (m, 1H, CHNH), 4.9 (d, 1H, NH), 6.7-6.8 (m, 3H, Ar),

### Synthesis of (2S,4S)-2-tert-butoxycarbonylamino-4-[4-methoxy-3-(3-methoxy propoxy)benzyl]-5-methylhexanoic acid (VIc) from racemic (R,R)-(S,S)-2-acetylamino-4-[4-methoxy-3-(3-methoxypropoxy)benzyl]-5-methylhexanoic acid (VIc) via enzymatic resolution

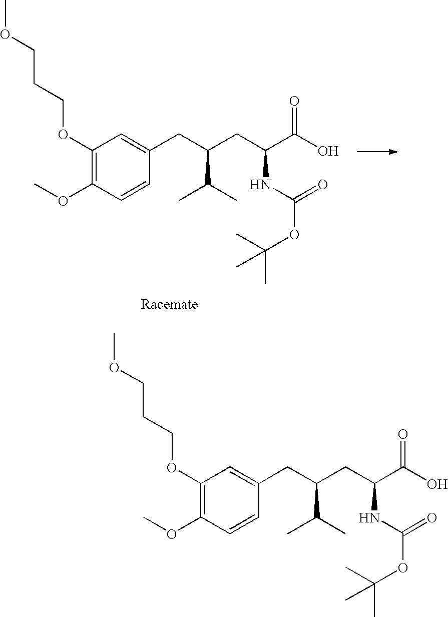

Racemic (R,R)-(S,S)-2-acetylamino-4-[4-methoxy-3-(3-methoxypropoxy)benzyl]-5-methylhexanoic acid (VIc) (80 mg, 0.2 mmol) is suspended in an aqueous lithium hydroxide solution (0.12 M, 2 mL) and the pH adjusted to 9.0 with aqueous acetic acid (10% w/w). Pig kidney acylase (25 mg) is added and the suspension is stirred at 38° C. for 48 h. At 41% conversion the pH is adjusted to 1 with aqueous hydrochloric acid (1 M, 1 mL) and the aqueous layer extracted with dichloromethane (3×5 mL). Decolourizing charcoal (110 mg) is added and the suspension filtered through a 1 μm filter membrane. The pH is adjusted to 8-9 (1M NaOH; 0.9 mL, then 0.1M HCl; 0.4 mL). Boc anhydride (30 mg, 0.14 mmol) and methanol (5 mL) are charged and the reaction stirred at room temperature overnight. The solvent is removed under vacuum and citric acid (0.5 M, 5 mL) is added (pH 2). The aqueous layer is extracted with dichloromethane (3×5 mL) and the combined organic layers dried over MgSO₄. The dried solution is filtered and concentrated to give (2S,4S)-2-tert-butoxycarbonylamino-4-[4-methoxy-3-(3-methoxypropoxy)benzyl]-5-methylhexanoic acid (VIc) as a single enantiomer as determined by chiral HPLC with a 98% d.e.

(2S,4S)-2-tert-butoxycarbonylamino-4-[4-methoxy-3-(3-methoxypropoxy)benzyl]-5-methylhexanoic acid:

¹H NMR (CDCl₃) 0.9 (m, 6H, CH(CH₃)₂), 1.4 (s, 9H, Boc), 1.6 (dd, 2H, CH₂), 1.7-1.8 (m, 2H, 2×CH), 2.1 (m, 2H, CH₂), 2.4 (m, 2H, ArCH₂), 3.4 (s, 3H, CH₃O), 3.6 (m, 2H, CH₂O), 3.8 (s, 3H, CH₃O), 4.1-4.2 (m, 2H, CH₂O), 4.3 (m, 1H, CHNH), 4.9 (d, 1H, NH), 6.7-6.8 (m, 3H, Ar).

1. A method for preparing a compound of the formula (VI) 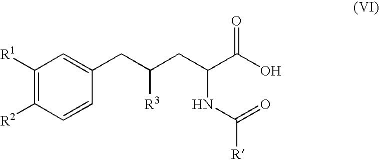
wherein
R¹ is halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof;
said method comprising hydrogenation of a pyrone compound of formula (V) 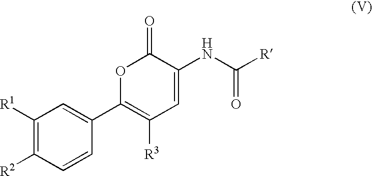
wherein R¹, R², R³, R⁴ and R′ are as defined for formula (VI), or a salt thereof, to effect ring opening.

2. The process according to claim 1 wherein R¹ is C₁₋₄alkoxy-C₁₋₄alkyloxy.

3. The process according to claim 1 wherein R² is C₁₋₄alkoxy.

4. The process according to claim 1 wherein R³ is branched C₃₋₆alkyl.

5. The process according to claim 1 wherein R′ is C₁₋₆alkyl or phenyl.

6. The process according to claim 1 wherein the compound of formula (VI) has the following stereochemistry:

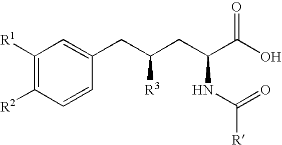

7. A method for preparing a compound of the formula (VI) 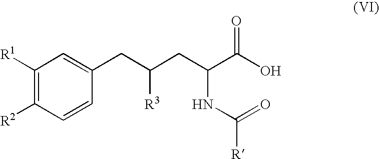
wherein
R¹ is halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof;
said method comprising hydrogenation of a pyrone compound of formula (V′) 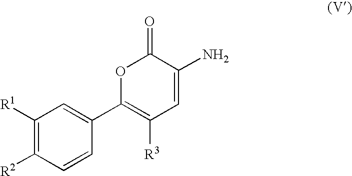
wherein R¹, R², R³, R⁴ and R′ are as defined for formula (VI), or a salt thereof, to effect ring opening.

8. The process according to claim 7 wherein R¹ is C₁₋₄alkoxy-C₁₋₄alkyloxy.

9. The process according to claim 7 wherein R² is C₁₋₄alkoxy.

10. The process according to claim 7 wherein R³ is branched C₃₋₆alkyl.

11. The process according to claim 7 wherein R′ is C₁₋₆alkyl or phenyl.

12. The process according to claim 7 wherein the compound of formula (VI) has the following stereochemistry:

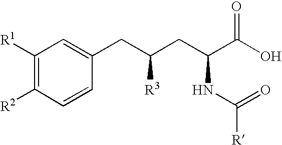

13. A compound of formula (V): 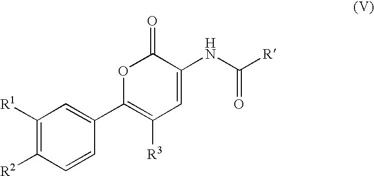
wherein
R¹ is halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl, preferably C₁₋₆alkyl or phenyl;
or a salt thereof.

14. The compound according to claim 13 having the following structure:

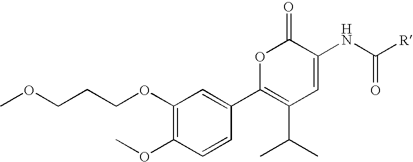

15. A compound of formula (V′): 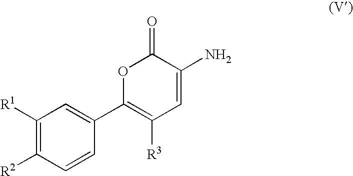
wherein
R¹ is halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl;
or a salt thereof.

16. The compound according to claim 15 having the following structure:

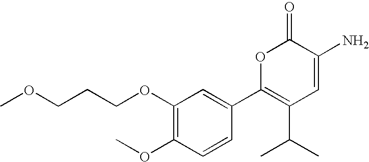

17. A method for preparing a compound of formula (V) 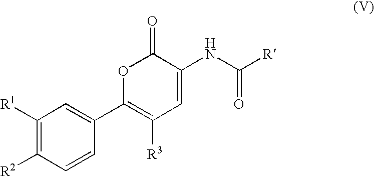
wherein R¹ is halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₁₄alkyl, OH, C₁₋₄alkylamino, di-C₁₋₄alkylamino, halogen and/or by trifluoromethyl, preferably C₁₋₆alkyl or phenyl; or a salt thereof,
said method comprising reacting an enamine compound of formula (III) 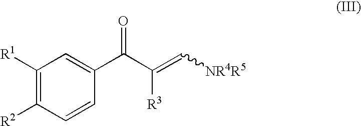
wherein R¹, R² and R³ are as defined for a compound of formula (V), R⁴ and R⁵ are independently C₁₋₆alkyl, preferably methyl or ethyl; or a salt thereof, with an amido glycine derivative of formula (IV) or (IV′) or a tautomer of (IV′) 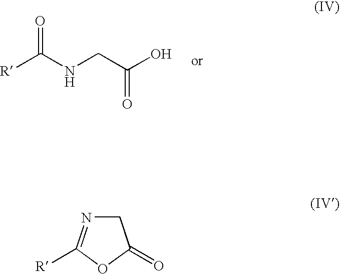
wherein R′ is as defined for a compound of formula (V); or a salt thereof to effect the ring closure to form a pyrone moiety.

18. The method according to claim 17 wherein the amido glycine derivative of formula (IV) is hippuric acid or N-acetylglycine.

19. The method according to claim 17 wherein the conversion takes place in the presence of an acid anhydride such as acetic anhydride.

20. A compound of formula (III): 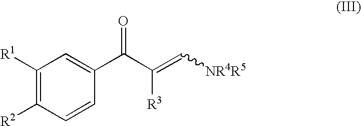
wherein
R¹ is halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl; and
R⁴ and R⁵ are independently C₁₋₆alkyl, preferably both methyl or ethyl; or a salt thereof.

21. The compound according to claim 20 having the following structure:

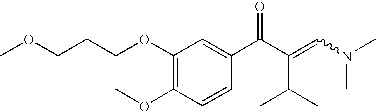

22. A method for preparing a compound of formula (V) 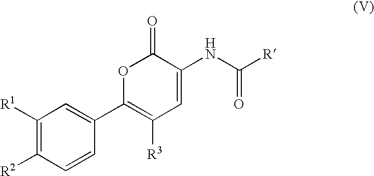
wherein R¹ is halogen, hydroxyl, C₁₋₆halogenalkyl, C₁₋₆alkoxy-C₁₋₆alkyloxy or C₁₋₆alkoxy-C₁₋₆alkyl, preferably C₁₋₄alkoxy-C₁₋₄alkyloxy;
R² is hydrogen, halogen, hydroxyl, C₁₋₄alkyl or C₁₋₄alkoxy, preferably C₁₋₄alkoxy;
R³ is C₁₋₇alkyl or C₃₋₈cycloalkyl, preferably branched C₃₋₆alkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl, preferably C₁₋₆alkyl or phenyl; or a salt thereof, said method comprising:
a) reacting an aryl ketone of formula (I) 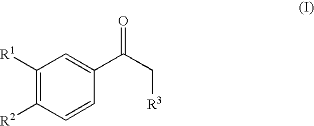
wherein R¹, R² and R³ are as defined for a compound of formula (V), or a salt thereof, with an amine of formula (II) 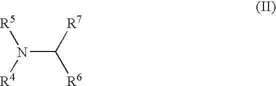
wherein R⁴ and R⁵ are independently C₁₋₆alkyl, preferably both methyl or ethyl; R⁶ and R⁷ are independently NR⁴R⁵ or O—C₁₋₆alkyl, or a salt thereof;
b) followed by reaction with an amido glycine derivative of formula (IV) or (IV′) or a tautomer of (IV′) 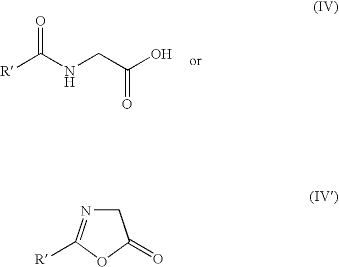
wherein R′ is as defined for a compound of formula (V)

23. The process of claim 22 wherein R⁶ and R⁷ are both O—C₁₋₆alkyl

24. The process of claim 22, wherein the amine of formula (II) is selected from the group consisting of Bredereck's reagent (tert-butoxybis(dimethylamino)methane), metoxybis(dimethylamino)methane, tris(dimethylamino) methane and dimethylformamidedimethylacetate, most preferably dimethylformamidedimethylacetate.

25. A method for preparing a compound of formula (VI) 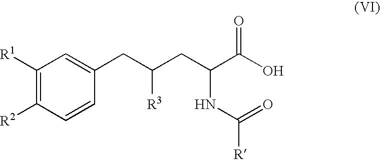
wherein
R¹ is halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof;
said method comprising one or more of the following steps either individually or in any combination:
the manufacture of a compound of the formula V according to claim 17, or a salt thereof, and
the manufacture of a compound of the formula VI according to claim 1, or a salt thereof.

26. A method for preparing a 2(S),4(S),5(S),7(S)-2,7-dialkyl-4-hydroxy-5-amino-8-aryl-octanoyl amide derivative having renin inhibitory activity such as aliskiren
said method comprising one or more of the following steps either individually or in any combination:
the manufacture of a compound of the formula V according to claim 17, or a salt thereof, and
the manufacture of a compound of the formula VI according to claim 1, or a salt thereof.

27. The method according to claim 25 comprising
the manufacture of a compound of the formula V according to claim 17, or a salt thereof.

28. The method according to 25 comprising
the manufacture of a compound of the formula VI according to claim 1, or a salt thereof.

29. A method for preparing a compound of formula (VI) 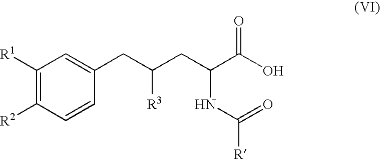
wherein
R¹ is halogen, hydroxyl, C₁₋₆halogenalkyl, C₁₋₆alkoxy-C₁₋₆alkyloxy or C₁₋₆alkoxy-C₁₋₆alkyl;
R² is hydrogen, halogen, hydroxyl, C₁₋₄alkyl or C₁₋₄alkoxy;
R³ is C₁₋₇alkyl or C₃₋₈cycloalkyl; and
R′ is C₁₋₇alkyl, C₂₋₇alkenyl, C₃₋₈cycloalkyl, C₁₋₇alkoxy, phenyl or naphthyl-C₁₋₄alkyl each unsubstituted or mono-, di- or tri-substituted by C₁₋₄alkyl, O—C₁₋₄alkyl, OH, C₁₋₄alkylamino, di-C₁₋₄alkylamino, halogen and/or by trifluoromethyl;
or a salt thereof;
said method comprising one or more of the following steps either individually or in any combination:
the manufacture of a compound of the formula V according to claim 22, or a salt thereof, and
the manufacture of a compound of the formula VI according to claim 1, or a salt thereof.

30. A method for preparing a 2(S),4(S),5(S),7(S)-2,7-dialkyl-4-hydroxy-5-amino-8-aryl-octanoyl amide derivative having renin inhibitory activity such as aliskiren
said method comprising one or more of the following steps either individually or in any combination:
the manufacture of a compound of the formula V according to claim 22, or a salt thereof, and
the manufacture of a compound of the formula VI according to claim 1, or a salt thereof.

31. The method according to claim 29 comprising
the manufacture of a compound of the formula V according to claim 22, or a salt thereof.

32. The method according to any claim 29 comprising
the manufacture of a compound of the formula VI according to claim 1, or a salt thereof.
